(19) United States
(12) Patent Application Publication
(10) Pub. No.: US 2004/0086579 A1
(43) Pub. Date: May 6, 2004

(54) Dietary supplement comprising parthenolide

(76) Inventors: James W. Higgins, Rothschild, WI (US); Russell W. Mitchell, Schofield, WI (US); Stephen C. Roberts, Minnetonka, MN (US)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 10/457,041

(22) Filed: Jun. 6, 2003

Related U.S. Application Data
(63) Continuation-in-part of application No. 10/288,610, filed on Nov. 5, 2002, now abandoned.

(51) Int. Cl. A61K 35/78; A61K 31/366

(52) U.S. Cl. 424/729; 424/756; 514/453

(57) ABSTRACT

An improved method for administering parthenolide and/or feverfew extract as a dietary supplement is provided, wherein an oral dosage composition comprising a dietary supplemental amount of parthenolide in a predetermined dosage amount of at least about 0.05 mg of parthenolide is orally administered to a patient. At least a portion of the administered oral dosage composition is retained by the patient in the oral cavity for a time sufficient to allow absorption of parthenolide by oral mucosal tissues. Compositions particularly suitable for oral mucosal administration of a parthenolide dietary supplement are also provided.

This application is a continuation-in-part of U.S. patent application Ser. No. 10/288,610, filed Nov. 5, 2002, entitled “COMPOSITIONS AND METHODS OF TREATMENT TO ALLEVIATE OR PREVENT MIGRAINOUS HEADACHES AND THEIR ASSOCIATED SYMPTOMS”, which application is incorporated herein by reference in its entirety.

### FIELD OF THE INVENTION

This invention relates to the field of natural dietary supplements. It is directed particularly towards the supplementation of diets, and most particularly towards supplementation of the diet with parthenolide.

### BACKGROUND OF THE INVENTION

Dietary supplements are materials provided to animals, typically multi-vitamin and mineral supplements, for treating specific medical conditions and as general nutritional supplements to promote and maintain good health of animals, particularly mammals. In particular, compositions and methods for optimizing the general health of both men and women by supplementing the daily diet with specific and multi-vitamin compositions. Dietary supplements are typically administered for ready consumption by the user, either as a combination with food or in a delivery vehicle suitable for swallowing by the user.

U.S. Pat. No. 6,475,511 to Gohlke, et al. discloses a dietary supplement for mammalian consumption, and particularly human consumption, for the purpose of stimulating the immune system, inhibiting infection and increasing tissue repair and healing. Comprising colostrum, lactoferrin, and with modified citrus pectin as an optional component, the dietary supplement is administered in ‘mucosal delivery format’: a dosage form that promotes effective absorption through the lining of the oral cavity.

U.S. Pat. No. 6,319,510 to Yates discloses a gum pad for the topical or systemic delivery of a wide range of pharmaceutical and nutritional agents. The gum pad is a laminate composed of: (a) a synthetic base or backing layer which is configured to be held in place on the gingiva (gums) in the mouth; (b) an intermediate, reservoir layer for containing medication therein; and (c) a semi-permeable outer layer facing outwardly toward oral mucosal tissues in the mouth which will allow saliva to enter and dissolve the medication in the reservoir layer into solution and pass the diffused saliva-medication solution outwardly to the oral mucosal tissues. The backing layer is placed on the gum so that the semi-permeable outer layer faces outwardly toward the buccal mucosa. Saliva enters the semi-permeable layer and dissolves the medication in the reservoir layer, then diffuses outwardly through the semi-permeable layer to the mucosal tissues in the mouth where it is readily absorbed into the circulatory system. Plants or plant components are also described as being capable of being delivered by the gum pad, “including those from garlic, ginkgo biloba, kava kava, noni, ginseng, saw palmetto, milk thistle, stinging nettle, eucalyptus, aloe vera, feverfew, nasturtium, Ma Huang, and echinacea.”

Feverfew is an herb that is widely available and has been investigated in modem times. Historically, feverfew is known to have been used in the treatment of fevers, from whence it derives its name, and also in rheumatic conditions. Feverfew is used in homeopathic remedies, but homeopathy recognizes no role for feverfew in the treatment of headaches. An authoritative homeopathic text is “A Dictionary Of Practical Materia Medica” by John Henry Clarke, M.D., recognized as such by the United States Food and Drug Administration, (see the “Compliance Policy Guide: Conditions Under Which Homeopathic Drugs may be Marketed” http ://www.fda.gov/ora/compliance ref/cpg/cpgdrg/cpg400-400.html). The Clarke text gives no indication for the use of feverfew in the treatment of headaches. This same text defines the appropriate preparation of feverfew as “a tincture of fresh leaves.” A tincture is a concentrated herbal extract prepared by soaking an herb in alcohol for an extended period of time. The result is an alcoholic extract referred to by homeopathic practitioners as the “mother tincture.” This “mother tincture” is then subject to numerous serial dilutions with the resulting homeopathic drug being extremely dilute. Classic homeopathic remedies do not rely on any effect from the substance first contained in the starting material (“mother tincture” in this case) and often statistically contain virtually no actual molecules of the original substance (feverfew). Instead, these remedies rely on the “imprint” or “energy” of the original substance to exert an effect. Consistent with the precepts of homeopathy, the remedy thus prepared is felt to become increasingly potent, indeed stronger and more effective, as it becomes more and more dilute. Some of these homeopathic remedies may have been administered sublingually, or may yet be administered sublingually by those presently adherent to the practice. Such purported remedies would certainly contain less than 0.01 mg/ml of parthenolide. There is little to no clinical support for any of the multitude of homeopathic remedies. Thus it is not surprising but still noteworthy that no clinical evidence exists for the effective use of feverfew in homeopathic medicine as a treatment for headache, especially as homeopathy does not recognize nor endorse this use. The amounts of supplement remaining in the homeopathic product, if not zero, are still substantially less than would be considered needful for general and beneficial supplementation of the diet.

Herbal medicine, as a field distinct from classical homeopathy, has in fact recognized the potential value of feverfew in the prophylactic (preventative) treatment of migraine. Fresh feverfew leaves have sometimes been chewed by subjects wishing to rid themselves of migraine. However, a common adverse effect reported by those who have used this technique is the generation sores in the mouth and sensitization of oral tissues. Additionally, many patients find this mode of administration to be crude and unpleasant.

In addition to raw leaves, feverfew tablets or capsules have been and are employed by practitioners of herbal medicine. These are widely available in any “health food store” for purchase by the general public. The PDR for Herbal Medicines lists migraine, arthritis, rheumatic diseases and allergies as the indications for feverfew usage (PDR for Herbal Medicines, Thompson Medical Economics, Second Edition, Feverfew, 306-309, 2000.) Several studies published in leading medical journals, including “Lancet” (Murphy, J J Lancet Jul. 23, 1988;2(8604): 189-92), and “The British Medical Journal” (Johnson, E S British Medical Journal Aug. 31, 1985;291(6495):569-73), have suggested a potential role for feverfew in reducing the incidence and/or severity of migraines. The Murphy study administered one capsule of feverfew leaves to be swallowed by the patient, wherein each capsule contained about 2.19 micromoles of parthenolide (about 0.5 mg). The Johnson study administered two capsules of freeze dried feverfew powder every morning. The daily dose was therefore 50 mg feverfew. The parthenolide content of this feverfew powder was not reported. Patients reported a reduction in the number and/or severity of migraine attacks, with no side effects reported by either study. Most recently however, several systemic reviews of feverfew use in the prevention of migraine have been published (Vogler B K, “Feverfew as a preventive treatment for migraine: a systematic review.” Cephalalgia December 1998;18(10):704-8) and (Pittler M H, “Feverfew for preventing migraine.” Cochrane Database Syst Rev 2000;(3):CD002286) both of which reviews concluded that the efficacy of feverfew for the prevention of migraine “has not been established beyond reasonable doubt.” Pittler also noted that “the trial with the highest methodological quality, which was also among the largest, found no significant difference between feverfew and placebo.” Most clinicians in the United States do not consider feverfew an effective prophylactic treatment for migraine and as such do not endorse its use. Feverfew has not been used for the acute relief of migraine attacks. Each of the studies investigated only its prophylactic use. The use of feverfew acutely, for relief of headaches once they have begun, has not been studied and there is no scientific literature that directly suggests that it might be effective. In addition, the prophylactic effect is not said to be noticeable for some number of weeks (2-12) after having first initiated use of feverfew, regardless of the form of feverfew employed (tablets, leaves, etc.). Recommended dosages of feverfew tablets or capsules are 200 to 250 mg one to three times daily, there being no suggestion that alternate routes of administration might prove beneficial.

A product currently available on the market is sold under the name MigraSpray®, which is stated to be a patented over the counter homeopathic drug intended to be a comprehensive approach for the treatment and prevention of migraine headaches. MigraSpray contains the active ingredients feverfew, polyporus, goldenseal and dandelion. MigraSpray is sprayed under the tongue (sublingual administration), which promotes enhanced bioavailability and rapid absorption by directly entering the bloodstream through the mucous membrane avoiding degradation from exposure to the gastrointestinal tract and liver. Product literature claims that this sublingual delivery system allows MigraSpray to provide rapid relief from migraine headache pain and other associated symptoms in an average of less than 7 minutes. However, this product, like other products sold as “homeopathic” treatments, delivers an extremely low dosage of feverfew. The amount of parthenolide reported to be present in this composition is 0.0112 mg/dose.

Additionally, it is noteworthy that the content of parthenolide in feverfew may vary to a great extent depending on the particular variety of Tanacetum parthenium plant grown, and also the manner of processing the feverfew herb. Parthenolide has been found to be unstable and sensitive to processing. Thus, the collection and processing steps carried out incorporating feverfew into a product may reduce or destroy the parthenolide content of the feverfew. Without a standardization of the parthenolide content of feverfew used in the process and careful control of the manufacturing process, great inconsistency is observed in parthenolide content from batch to batch of product.

### SUMMARY OF THE INVENTION

The present invention provides an improved method for administering parthenolide and/or feverfew extract as a dietary supplement comprising providing an oral dosage composition comprising a dietary supplemental amount of parthenolide in a predetermined dosage amount of at least about 0.05 mg of parthenolide. This composition is orally administered to a patient. At least a portion of the administered oral dosage composition is retained by the patient in the oral cavity for a time sufficient to allow absorption of parthenolide by oral mucosal tissues. Preferably, the composition is retained in the mouth for at least about 30 seconds, and more preferably at least about 60 seconds. Most preferably, the composition is retained sublingually in the oral cavity for at least about 30 seconds, and more preferably at least about 60 seconds. This mode of administration has been found to promote efficient absorption of the parthenolide and/or feverfew extract supplement through the oral cavity's epithelial lining. Compositions particularly suitable for oral mucosal administration of a parthenolide dietary supplement are also provided. Compositions of the invention that are preferred also render the dietary supplement of the invention particularly adaptable to self-monitored dosages, and are especially appropriate for regimes of self administration.

### DETAILED DESCRIPTION

Previous modes of administration of parthenolide have generally administered parthenolide and/or feverfew through the GI tract, where it is believed to be rapidly degraded by the acidic and enzymatic conditions of the stomach and intestine. Other administration attempts have utilized far too small an amount of parthenolide to be effective as a dietary supplement. The practice of chewing feverfew leaves suffers from a number of disadvantages, including widely varied parthenolide content in any particular quantity of feverfew leaves. Because the amount of parthenolide in leaves cannot be standardized among a sufficient amount of leaves, it is not possible for a known quantity of parthenolide to be administered in this manner. Further, studies have shown that in some cases, depending on various production factors, samples of feverfew leaves have been found to contain no parthenolide whatsoever.

Compositions of the present invention comprise a dietary supplemental amount of parthenolide that is in a predetermined dosage amount of at least about 0.05 mg of parthenolide. Because the dosage is predetermined, the user can rely on a standard and reproducible quantity of the desired active to be administered. Thus supplement, parthenolide can therefore be provided in a form that provides ready self monitoring and control of treatment regimes. Preferably, the composition comprises from about 0.05 to about 50 mg, and more preferably from about 1 to about 30 mg of parthenolide in each dosage unit.

Parthenolide is a sesquiterpene lactone that may be obtained from a number of sources. A preferred source of parthenolide is by extraction from the feverfew plant (Tanaecetum parthenium), which is also known, for example, as Chrysanthemum parthenium, Chrisanthemum parthenium, Pyrethrum parthenium, Tanacete parthenii herba or folium, Matricaria parthenoides, Matricaria parthenium, Leucanthemum parthenium, Matricaria parthenium, Spanish pellitory, Featherfew, Featherfoil, feather-fully, and by a number of common names, various of which are used throughout the world (Midsummer daisy, Bachelor's buttons, Altamisa, nosebleed, flirtwort, ague plant, devil daisy, feddygen fenyw (Welsh), maid's weed, Missouri snakeroot, mutterkaut (German), prairie-dock, vetter-voo, wild chamomile, grande camomille (French), Santa Maria (Spain), febrifuge plant.) The extract may be obtained by techniques known in the art using solvents such as petroleum spirits or polar organic solvents. See U.S. Pat. No. 5,384,121 to Rhodes, and also WO 94 06800; EP 0 553 658; WO 92 11857; GB 2,166,952; EP 98 041; WO 98 39018.

Compositions of the present invention may comprise additional ingredients providing nutritional or organoleptic benefit to the ultimate composition. Thus, additional ingredients such as sesquiterpene lactones, vitamins, mineral or other ingredients desirable for supplementing the dietary needs and/or imparting healthful benefits to the patient in need thereof are contemplated.

The extract of the feverfew plant contains parthenolide, and may additionally contain other components such as Polyynes, Flavonoids and Volatile oils including camphor, bomeol and others, each of which may contribute as a dietary supplement benefits of the composition disclosed herein. Feverfew also contains relatively large quantities of sesquiterpene lactones.

In addition to parthenolide, feverfew is known to contain the following non-ubiquitous chemicals: 1-Beta-hydroxyarbusculin, 10-Epicanin, 8-Beta-reynosin, Apigenin-7-glucoside, Chrysanthemolide, Chrysanthemonin, Chrysartemin-A, Chrysartemin-B, Cosmosiin, L-Bomeol, L-camphor, Mangoliolide, Reynosin, Santamarin, Tanaparthin, Tanaparthin-1-alpha, 4-alpha-epoxide, Tanaparthin-1-beta,4-beta-epoxide, tenetin 3-b-hydroxyparthenolide, seco-tanaparthenolide A, canin, artecanin, and balchanin.

Because feverfew extract may contain additional beneficial components, compositions comprising the extract of feverfew are generally preferred for use in the present invention as compared to compositions comprising a highly purified parthenolide that has been isolated from the additional components naturally occurring in feverfew extract. Preferred embodiments of the present invention use feverfew extract that has been standardized to contain a predetermined standardized parthenolide concentration of preferably not less than about 1.0%, and more preferably from about 1.2% to about 10%. Higher concentration parthenolide compositions may become readily available, which may advantageously reduce the amount of liquid required in the composition for delivery of the active to the user. While the source of parthenolide in compositions of the present invention is preferably feverfew as discussed above, it may alternatively be obtained from any number of other plant species, where it generally occurs in substantially lower concentrations. Such plant species include especially other members of the Compositae family, which include especially the many species of chrysanthemums, daisies, marigolds, chamomile, yarrow and aster. Parthenolide can also be obtained from tansy. Alternatively, parthenolide may be made by any appropriate synthetic route.

The composition to be used in the present invention may optionally comprise additional active ingredients. These active ingredients may also be provided as a dietary supplement or may provide other physical benefits, provided that the benefit of parthenolide is not adversely affected. In one aspect, preferably additional amounts of already present sesquiterpene lactones or additional sesquiterpene lactones are incorporated in the compositions of the present invention. Preferred such sesquiterpene lactones include especially those which are known to be contained in (naturally occur in) feverfew, such as 3-Beta-hydroxyparthenolide, seco-tanaparthenolide A, canin, artecanin, chrysanthemonin, chrysartemin A and B, santamarin and balchanin, as well as those occurring in other plant species such as encelin, leucanthin B, enhydrin, melampodin A, tenulin, confertiflorin, burrodin, psilostachyin A, costunolide, guaianolide, cinerenin, artemisinin, aristolactone, lactarorufin A, bilobalide, helenalin, furandiol. Sesquiterpene lactones in addition to parthenolide may be isolated from plants such as dandelion, burdock, butterburr, mugwort and sunflower plants, among others.

Compositions to be used in the present invention may optionally additionally comprise other naturally occurring components and extracts, including those as identified in the HPUS. Preferred additional components are extracts indicated for use in treatment of headaches, inflammation, nausea or anxiety. Particularly preferred additional components are the extracts of bay leaf and/or ginger and/or green tea, or the isolated components thereof. A particularly preferred isolated component of green tea is L-theanine.

Particularly preferred compositions of the present invention contain substantially no active ingredients other than those that are extractable from herbal sources. In a particularly preferred embodiment of the present invention, the compositions contain substantially no active ingredients other than those that are extractable from feverfew, ginger and green tea sources. In another particularly preferred embodiment, the compositions contain substantially no active ingredients other than those that are extractable from feverfew and ginger. Such compositions additionally may comprise non-pharmacologically active ingredients, such as thickeners, carrier liquids and flavorants. It has surprisingly been discovered that the use of only active ingredients that are extracted from herbs provide particular benefit to the user in being both effective in the treatment of migrainous headache, and also providing natural healing conditions particularly suited to the well being of patients. Surprisingly, these natural ingredients have been found to be effective in the indicated dosage ranges when administered in a sublingual regimen as described herein. Such compositions contain parthenolide in the amounts as discussed earlier, and preferably contain less than about 400 mg of any given natural active ingredient per dose.

In a particularly preferred embodiment, the composition to be used in the present invention additionally comprises ginger extract at a total administered amount preferably not exceeding about 400 mg, and more preferably not exceeding about 250 mg of ginger extract. Particularly preferred compositions comprise ginger extract as about 0.1-10% of the total composition.

In another particularly preferred embodiment, the liquid composition additionally comprises L-theanine, either as an isolated component or as a constituent of green tea extract, but in either case at a total administered amount preferably not exceeding about 400 mg of L-theanine. Particularly preferred compositions comprise L-theanine as about 0.1-10% of the total composition.

Organoleptically beneficial additives are also contemplated, such as components that contribute to the texture, color or flavor of the composition may also be incorporated in the present composition.

The compositions as described herein may further comprise suitable adjuvants, such as preservatives (for example, sodium benzoate, sorbic acid and esters of p-hydroxybenzoic acid), stabilizers, antibacterial agents (such as benzyl alcohol or methyl paraben), antioxidants (such as ascorbic acid or sodium bisulfite), chelating agents (such as ethylenediaminetetraacetic acid), buffers (such as acetates, citrates or phosphates), agents for the adjustment of tonicity (such as sodium chloride or dextrose), dyes, colorants, thickening agents, flavorants, sweetening agents, and suspending agents. For example, compositions of the present invention preferably comprise dextrose and/or sucrose as filler and sweetener, artificial or natural flavors such as fruit or vanilla or chocolate flavoring,

In a particularly preferred embodiment of the present invention, the compositions of the present invention are provided in combination with a mucosal permeation enhancer appropriate for enhancing the mucosal absorption of the composition employed. The mucosal permeation enhancer preferably comprises azone, sodium glycholate, sodium cholate, sodium taurocholate, sodium taurocholate plus EDTA, deoxycholate, sodium lauryl sulfate, lauric acid, ethanol, lysophosphatidyl choline, polysorbate 80, cyclodextrin, cetylpyridinium chloride, cetyltrimethylammonium bromide, benzalkonium chloride, sodium salicylate, sodium EDTA, aprotinin, dextran sulfate, linoleic acid, labrafil, transcutol, urea, methoxysalicylate, POE 23 lauryl ether, various surfactants and other mucosal permeation enhancers and combinations thereof. Most preferably, the mucosal permeation enhancer comprises sodium lauryl sulfate.

In a particularly preferred embodiment of the present invention, the compositions of the present invention are provided at a pH of from about 2.0 to about 6.5, more preferably at a pH of from about 2.5 to about 6.0, and more preferably at a pH of from about 3 to about 5. Surprisingly, it has been found that compositions having a pH of the range indicated above are much more effective than compositions outside of the indicated range. Various pH adjusters may be used to adjust the pH of the composition to the desired level. Examples of suitable pH adjusters include hydrochloric acid, citric acid, phosphoric acid, acetic acid, tartaric acid, sodium hydroxide, potassium hydroxide, sodium carbonate, sodium hydrogen carbonate, boric acid, sodium borate, and the like. Preferably, the pH of the composition is adjusted to be acidic using ascorbic acid.

Preferably, the composition is buffered by a pharmaceutically acceptable buffer. Examples of buffering agents include borate buffers, citrate buffers, phosphate buffers, tartarate buffers, acetate buffers, carbonate buffers, and amino acid salts, etc. Most preferably, the buffer is sodium citrate.

Preferably, the liquid composition comprises 0.05%-10% feverfew extract, 0.1-10% ginger extract, 0.1-10% L-theanine, and 10-98% water. Preferably, the feverfew extract has a standardized parthenolide concentration of not less than about 1.0%, and more preferably from about 1.2% to about 10%.

Compositions of the present invention are preferably provided in the form of a liquid. Such compositions are particularly suitable for use as a dietary supplement where the absorption of active materials for use by the body of the user in a rapid manner is desired.

The liquid compositions as described herein are formulated using a carrier liquid appropriate for administration to the sublingual region of the mouth. The carrier liquid preferably is selected from water, alcohol, polyethylene glycols, glycerin, propylene glycol, and mixtures thereof. Most preferably the carrier liquid comprises water.

It has additionally been found that the efficacy of the composition in enhanced when the composition has a viscosity greater than water, and more preferably when the composition has a viscosity greater than about 100 cP. Compositions having higher viscosity have been found to enable the patient to better establish and maintain contact of the composition with the sublingual area. Thickening agents are preferably incorporated in compositions of the present invention. The thickening agent preferably assists in retention of the liquid composition sublingually for a time sufficient to allow absorption of the active ingredients in by the patient.

Thickening agents are particularly desirable in sublingual applications, as a more viscous agent is more easily retained in the proper area. A more viscous agent further reduces the user's involuntary impulse to swallow, in this case perhaps prematurely. Thus, the thickening agent may assist in providing sublingual liquid retention for a time appropriate for proper absorption of the active ingredient by the patient, and also thereby may improve the clinical efficacy of the composition. Any appropriate thickening agent may be used in the composition of the present invention. Preferred such thickening agents include agar, alginate, carageenan, carboxymethylcellulose, cellulose, chitosan, corn starch, Danish agar, dextrin, furcelleran, galactomannans, gelatin, gellan gum, guar gum, gum acacia, gum arabic, gum ghatti, gum tragacanth, hydroxypropyl methylcellulose, karaya gum, methylcellulose, polyvinyl alcohol, carboxyvinyl polymer, polyvinylpyrrolidone, hyaluronic acid and salts thereof, modified starches, mucilage, pectin, potato starch, rice starch, starch, tara gum, vegetable starch, wheat starch, and xanthan gum and combinations thereof.

Most preferably, the compositions of the present invention have a viscosity that is from about 100 cP (somewhat lower than the viscosity of Olive Oil) to about 50,000 (i.e. the viscosity of molasses), and more preferably from about 500 cP (the viscosity of SAE #10 motor oil) to about 5000 cP (approximately the viscosity of Corn Syrup), all measured at 25° C.

Preferably, the composition is provided as an aqueous composition.

For purposes of the present invention a composition is considered to be “aqueous” if it contains water in an amount sufficient to act as the solvent for the parthenolide and/or feverfew solute. Preferably, water is present as the majority component of the composition. Most preferably, water is present at an amount of from about 30% to about 99.9% of the total composition, and more preferably from about 50% to about 99% of the total composition. The compositions of the present invention are preferably aqueous because it has been found that such compositions readily deliver the desired active ingredient systemically to the patient in a rapid manner. Additionally, aqueous compositions are generally more acceptable to the patient organoleptically during administration of the composition sublingually. Small amounts of oil may be incorporated in the composition as some trace amount of oil remains as a component of an herbal extract, and also particularly when such incorporated oils are oils that enhance the flavor of the composition. Preferably, the composition comprises no more than about 5%, and more preferably no more than about 2% oil by weight.

In one aspect of the present invention, convenient systems for administration of parthenolide, and as another embodiment, feverfew extract, are provided wherein compositions are provided in a unit dose applicator for sublingual administration. More particularly, a unit dose applicator and composition for sublingual treatment of patients is provided comprising a dispenser for dispensing liquids having a reservoir and a delivery spout. The dispenser has a liquid capacity of about 0.1 to about 10 mls. This dispenser is provided with a liquid composition disposed therein. The composition comprises parthenolide in an amount not exceeding about 1.0 mg. In another embodiment, the liquid composition in the unit dose dispenser comprises feverfew extract in an amount not exceeding about 40 mg.

Liquid compositions as described herein may be sublingually administered using any appropriate technique, such as by use of a medicine dropper, syringe, vial, or the like. Most preferably, the aqueous composition is administered in a controlled manner as a flow of liquid, rather than as a spray. A flowing liquid dispenser provides benefits of controlled delivery of the liquid to the desired position in the mouth, enhancing the likelihood that the composition to be dispensed is properly delivered. Preferably, the composition is administered using a unit dose applicator that is a dispenser having a reservoir and a delivery spout and having a liquid capacity of about 0.1 to about 10 mls. In a preferred embodiment, the unit dose applicator is provided as a dispenser having parthenolide in an amount not exceeding about 1.0 mg, or other limited quantities as discussed above.

In an alternative embodiment of the present invention, compositions of the present invention are provided in solid or semisolid form, in a format suitable for retaining in the oral cavity for period of time sufficient to allow absorption of parthenolide by oral mucosal tissues. Preferably, the solid or semisolid form is designed to facilitate retention of the composition in the oral cavity for a time longer than 30 seconds to allow delivery of the active material to the mucosal tissues of the mouth. Examples of oral dosage forms that promote absorption of the dietary supplement's components within the oral cavity are those that encourage retention of the dose within the oral cavity for an extended period, or discourage swallowing of the dose. Dosage forms that are chewable or that are slow dissolving as in a cough drop or hard candy are examples; they may be additionally designed to encourage salivation. Such dosage forms include lozenges, particularly chewable lozenges, slow dissolving lozenges, chewable tablets and chewable gums. The addition of natural or artificial flavoring also encourages retention of the dosage form within the mouth, particularly with children, so that there is greater transfer of the active components through the lining of the oral cavity and into the bloodstream and/or the lymphatic system. The physical size and consistency of the dosage form may also be adapted to prevent premature swallowing of the delivered dose. A period of time of from about 30 seconds to ten minutes is recommended for which the dose should remain in the mouth for effective absorption, with better effects being observed at the longer retention times. Larger chewable forms are appropriate for animals that would otherwise be likely to swallow such foodstuff with little mastication.

Lozenges, in contrast to pills or capsules, provide for delivery of the active ingredients of the dietary supplement so that they can be absorbed through the oral mucosal surface. In particular, the lozenges of the invention are able to enhance the benefits associated with absorption of appropriate constituents through the oral epithelial mucosa and into the underlying lymphatic system, for they are designed to be dissolved slowly in the mouth and they may also be chewable: such lozenges are therefore a preferred composition format. By using a cold-pressing technique to manufacture the lozenges, heat degradation of sensitive biological components is minimized. Lozenges are also preferable to hard-pressed tablets or capsules, because the latter do not dissolve until exposed to the gastric juices of the stomach. These strongly acidic juices degrade the sesquiterpene lactone active agents to be delivered to the patient in need thereof. Preferably, a lozenge is formed by cold pressing the ingredients into a chewable lozenge of hardness 14 to 44 Kp.

Solid and semisolid forms of the composition of the present invention may be readily prepared by the routineer in the field, by consulting with established formularies and substituting the indicated active ingredients as taught herein.

Compositions of the present invention are administered so that they reside in the mouth for a time prior to consumption by the user. The residence time in the oral cavity has been found to be important is delivering an immediate quantity active dietary supplement without requiring the composition to go through the GI tract.

Compositions of the present invention are administered as indicated above, preferably once or twice or more often per day, as needed, to provide the suggested dietary supplement. More preferably, the composition comprises the following ingredients that is taken as a nutritional supplement one to five times per day:

Most preferably, the composition is administered as a first sublingual application of a first composition comprising parthenolide, which first composition is held in place under the tongue for a predetermined time, preferably about 30 seconds, or more preferably about 60 seconds or more, after which the composition is swallowed. Most preferably, the composition is circulated or “swished” around the mouth by the patient prior to swallowing. Surprisingly, this apparently minor addition to the procedure noticeably increases the effect of the composition in the treatment. A second composition comprising parthenolide is then applied and held under the tongue for a predetermined time, preferably about 30 seconds, or more preferably about 60 seconds or more, after which the second composition also is swallowed. Again, preferably the composition is circulated or “swished” around the mouth by the patient prior to swallowing.

As an alternative to the unit dose applicator preferentially utilized as described above, a bottle designed so as to dispense only a certain, measured dose may be used. Alternatively, the composition may be provided in a conventional bottle with instructions to measure a dose, with or without a dedicated appliance for so doing (e.g. cup, syringe). Alternative delivery vessels that do not deliver premeasured quantities of liquid lack the advantages of convenience and higher probability of administration of the correct amount of the composition, but may be more economical than delivery of the composition using a unit dose system.

When delivered in the form of a chewable lozenge, the lozenge is chewed for 30 seconds to ten minutes to maximize absorption of the active ingredients through the lining of the oral cavity and their absorption into the blood and lymphatic system. Likewise, other solid and semisolid compositions are administered, preferably sublingually, in a manner to maximize absorption of the active ingredients through the lining of the oral cavity and their absorption into the blood and lymphatic system.

Dietary supplements of the present invention may have beneficial effects on the health and well being of patients particularly due to the biological impact that parthenolide and other sesquiterpene lactones, especially those containing an α-methylene-γ-lactone group, have demonstrated. Specifically, these compounds have been shown to possess activity against tumor growth and general inflammation. Specific sesquiterpene lactones have been asserted to have desired effects on the Nuclear factor (NF)-kB pathway, which is intimately involved in many disease states. The NF-kB pathway was first discovered in 1986. The pathway is central and is essential for basic immune response. It is also intimately involved in many disease states, including those of chronic inflammation (arthritis, inflammatory bowel disease, multiple sclerosis, etc.), those of acute, intermittent inflammation (migraine, asthma, etc.), those of auto-immune disease (lupus, fibromyalgia, autoimmune myocarditis, etc.), those of dysfunctional immune response (cancer, AIDS, etc.), those associated with infectious agents and parasites (Hepatitis B&C, H. pylori, malaria, tuberculosis, etc.), those associated with endocrine function (diabetes, pancreatitis, etc.) those associated with degenerative proceses (aging, alzheimers, etc.) those genetically mediated (muscular dystrophy, etc.), and those associated with trauma (heat shock, post-perfusion injury, restenosis after angioplasty, etc.) among many others.

Specific genes known to be regulated by NF-KB and implicated in disease include: Cyclin D1 (cancer); IL-8 (asthma); MCP1 (atherosclerosis); MMP9 (cancer, arthritis); c-Myc (cancer); 5′deiodinase (euthyroid sick syndrome); HIV LTR (AIDS); Bcl-xL (cancer); c-IAP2 (cancer); iNOS (septic shock); COX-2 (inflammation, colorectal cancer).

All patents, patent documents, and publications cited herein are incorporated by reference as if individually incorporated. Unless otherwise indicated, all parts and percentages are by weight. The foregoing detailed description has been given for clarity of understanding only. It will be appreciated that numerous modifications and variations of the invention are possible in light of the above teachings, and therefore the invention may be practiced otherwise than as particularly described.

What is claimed is:

1. A method of administering a dietary supplement comprising parthenolide, comprising a) providing an oral dosage composition comprising a dietary supplemental amount of parthenolide in a predetermined dosage amount of at least about 0.05 mg of parthenolide;
b) orally administering the oral dosage composition to a patient; and
c) retaining at least a portion of the administered oral dosage composition in the oral cavity for a time sufficient to allow absorption of parthenolide by oral mucosal tissues.

2. The method of claim 1, wherein the composition comprises from about 0.05 to about 50 mg of parthenolide in each dosage unit.

3. The method of claim 1, wherein the composition comprises from about 1 to about 30 mg of parthenolide in each dosage unit.

4. The method of claim 1, wherein said composition additionally comprises ginger extract.

5. The method of claim 1, wherein said composition additionally comprises green tea extract.

6. The method of claim 1, wherein said composition comprises additional sesquiterpene lactones.

7. The method of claim 1, wherein said composition additionally comprises a mucosal permeation enhancer.

8. The method of claim 1, wherein said composition has a pH of from about 2 to about 6.

9. The method of claim 1, wherein said composition has a pH of from about 3.0 to about 6.0.

10. The method of claim 1, wherein said composition contains substantially no active ingredients other than those that are extractable from herbal sources.

11. The method of claim 1, wherein said compositions contains substantially no active ingredients other than those that are extractable from feverfew, ginger and green tea sources.

12. The method of claim 10, wherein said composition contains less than about 400 mg of any given natural active ingredient per dose.

13. The method of claim 1, wherein the composition is retained sublingually.

14. The method of claim 1, wherein said composition is retained in the oral cavity for at least about 30 seconds prior to swallowing.

15. The method of claim 1, wherein said composition is retained in the oral cavity for at least about 60 seconds prior to swallowing.

16. The method of claim 1, wherein the composition is a liquid.

17. The method of claim 1, wherein the composition is an aqueous liquid.

18. The method of claim 1, wherein the liquid composition has a viscosity of from about 500 cP to about 5000 cP.

19. The method of claim 1, wherein the composition is first retained sublingually for at least about 30 seconds and then circulated about the mouth prior to swallowing.

20. The method of claim 1, wherein the composition is a solid or semisolid composition.

21. The method of claim 20, wherein the composition is selected from the group consisting of chewable lozenges, slow dissolving lozenges, chewable tablets and chewable gums.

22. A dietary composition for oral mucosal administration of parthenolide, which composition is selected from the group consisting of a liquid, hard lozenges for sucking on, a chewable lozenge, a chewable tablet or a chewable gum, the composition comprising a predetermined dosage amount of greater than about 1.0 mg of parthenolide.

23. A unit dose applicator and liquid composition for sublingual treatment of patients, comprising
a) a dispenser for dispensing liquids, said dispenser having a reservoir and a delivery spout, said dispenser having a liquid capacity of about 0.1 to about 10 mls, and
b) an aqueous liquid composition disposed within said dispenser, said composition comprising parthenolide in an amount greater than about 1.0 mg.
